(19) United States
(12) Patent Application Publication
(10) Pub. No.: US 2010/0129448 A1
(43) Pub. Date: May 27, 2010

(54) TOPICAL HYDROGEL COMPOSITION

(75) Inventor: James D. Talton, Gainesville, FL (US)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: Nanotherapeutics, Inc., Alachua, FL (US)

(21) Appl. No.: 12/542,925

(22) Filed: Aug. 18, 2009

Related U.S. Application Data
(60) Provisional application No. 61/089,568, filed on Aug. 18, 2008.

(51) Int. Cl. A61K 31/65; A61P 17/02; A61K 9/00

(52) U.S. Cl. 424/484; 514/153; 424/489

(57) ABSTRACT

Compositions for topical application to the skin and/or a wound are disclosed. In some embodiments, the compositions include a suspension or dispersion of particles of at least one poorly soluble drug chelated or otherwise complexed with a metal salt. The compositions may further contain at least one stabilizer, at least one excipient, and at least one physiologically acceptable carrier. Methods for making such compositions, pharmaceutical formulations containing such compositions, and methods of treatment utilizing such compositions are also disclosed.

This application claims benefit of priority to U.S. Provisional Application No. 61/089,568, filed Aug. 18, 2008, the contents of which are incorporated herein by reference.

### FIELD

The present disclosure relates to topical compositions for application to the skin and/or a wound. The present disclosure also relates to methods of treating the skin and/or wounds with such compositions, and methods for manufacturing such compositions.

### BACKGROUND

Wound dressings are designed with care so that their application does not increase insult or inflammation of a wound. One of the major determinants of wound healing involves keeping the wound wet, since dry dressings often retard healing. Factors such as thickness, diffusivity, occlusiveness, and osmotic pressure of a dressing impact the direction and rate of movement of gases and water across the membrane of the dressing.

The relative osmotic pressures of wounds, blood plasma, and cells determines the allocation of water between these sites. Electrolytes, glycerol and other compounds have been proposed for the improvement of wound remodeling and energy metabolism. In particular, glucose, pyruvate, alanine, and/or lactate have proven useful, at least partly because they function to increase the amount of energy available for use by cells (see, e.g., U.S. Pat. No. 5,238,684). Arginine has also been proposed as potentially useful for increasing the rate of wound closure (healing).

Within the last few years, many silver-based antimicrobial dressings have become available, such as Acticoat™ (Smith & Nephew, Largo, Fla.), Silverlon™ (Argentum, Lakemont, Ga.), and Silvasorb™ (Medline Industries, Inc, Mundelein, Ill.). However, because such dressings are relatively expensive, the standard of care is to change simple dressings daily or anti-microbial dressings weekly.

Several disadvantages have also been identified with silver-based dressings. For example, pre-clinical and clinical study data has suggested that: a) bacterial resistance to silver may occur; b) silver dissociation can be affected by the test medium used; c) differences in bactericidal activity may be a function of the bacterial strain used for testing; d) relatively high silver concentration may be needed due to the binding of silver ions to proteins and nucleic acids; e) rapid delivery of silver (i.e., rate of kill) may be a positive factor when considering prevention of silver resistance and biofilm formation; and f) silver may affect viable cells and thus, may be cytototoxic.

Argyria is also a possible side effect of silver-based wound dressings. Argyria is a rare dermatosis in which excessive administration and deposition of silver causes a permanent irreversible gray-blue discoloration of the skin or mucous membranes. The amount of discoloration typically depends on the route of silver delivery (i.e., oral or topical administration) and the body's ability to absorb and excrete the administered silver compound. Once silver particles are deposited within the skin and/or mucous membranes, they may remain immobile and may accumulate during the aging process.

Other forms of dermatitis and irritating reactions (e.g., contact dermatitis) may also arise from the use of silver-based bandages, and may be caused by ingredients of the base and/or the active ingredients. As a result, alternatives to silver topical treatments that provide anti-microbial effects, promote wound healing, and avoid unwanted side-effects have been sought in the art.

Some alternatives that have been explored are treatments utilizing tetracycline compounds. However, the stability of such compounds in solution is often poor. One method to improve the stability of tetracyclines is to include such compounds and their derivatives in an aqueous suspension/dispersion, wherein the aqueous phase of the suspension/dispersion contains the compounds in an amount greater than solubility of the compound permits. Doxycycline Monohydrate, for example, exhibits an aqueous solubility of less than 0.8 mg/ml at a pH greater than 6, and so is only very slightly soluble in water (Bogardus, J B, et al. 1979. J. Pharm Sci 68:188-94). In such compositions, however, relatively high concentrations of Doxycycline, e.g., greater than 0.1% by mass, lead to a gritty suspension that is not suitable for topical application to the skin or a wound. Accordingly, such compositions are generally administered to the body via some alternative route, such as orally.

One example of a suspension that is suitable for oral administration is Vibramycin® (Pfizer), which contains large Doxycycline particles in a viscous syrup. However, Vibramycin is not approved for local or topical use, and its pH is inappropriate for application to a wound. Moreover, the size of the particles in Vibramycin can lead to wound irritation if the suspension is applied topically.

Thus, there remains a need in the art for topical compositions and methods for the delivery of a tetracycline class compounds, such as Doxycycline, in high concentrations above the aqueous solubility of the compounds, and without the aforementioned problems. The present disclosure addresses this need by providing, for example, compositions comprising small particles of a poorly soluble drug, such as Doxycycline, chelated or otherwise complexed with a physiologically acceptable salt in a physiologically acceptable carrier, such as a hydrogel.

### SUMMARY

Disclosed herein are compositions for topical application to the skin and/or a wound. In one non-limiting embodiment, the compositions include a suspension or dispersion of particles of at least one poorly soluble drug chelated or otherwise complexed with a physiologically acceptable salt, such as a calcium salt. The compositions further contain at least one physiologically acceptable carrier, and optionally further contain at least one stabilizer and/or at least one excipient.

One non-limiting embodiment of the present disclosure is a composition for topical application to the skin or a wound comprising: a suspension or dispersion of particles of at least one tetracycline class compound complexed with a physiologically acceptable metal salt, wherein the composition further comprises at least one stabilizer, at least one excipient, and at least one physiologically acceptable carrier, and said particles have an average diameter less than or equal to about 100 μm.

Another non-limiting embodiment of the present disclosure is a composition for topical application to skin or a wound, comprising a suspension or dispersion of particles of at least one tetracycline class compound chelated to a physiologically acceptable calcium salt, wherein the composition further includes a carboxy-methyl-cellulose hydrogel, glycerol, water, and at least one pH stabilizer, and said particles have an average diameter less than or equal to about 100 μm.

A further non-limiting embodiment of the present disclosure is a method of making a composition for topical application to skin and/or a wound, the method comprising mixing particles of at least one tetracycline class compound with at least one physiologically acceptable metal salt to form metal-chelated particles, said particles having an average diameter less than or equal to about 100 μm; combining said metal-chelated particles with at least one physiologically acceptable carrier to form a suspension or dispersion of metal-chelated particles, and optionally combining at least one excipient and/or at least one stabilizer with said suspension or dispersion.

Also disclosed herein are pharmaceutical formulations that include the compositions disclosed herein, methods for making such compositions, and methods of treatment utilizing such compositions.

Additional objects and advantages of the present disclosure will be set forth in part in the description which follows, and in part will be obvious from the description, or may be learned by practice of the present disclosure. The objects and advantages of the present disclosure will be realized and attained by means of the elements and combinations particularly pointed out in the appended claims.

It is to be understood that both the foregoing general description and the following detailed description are exemplary and explanatory only and are not restrictive of the invention, as claimed.

### DETAILED DESCRIPTION

One aspect of the present disclosure relates to topical compositions for application to the skin and/or a wound. In general, the compositions disclosed herein include a suspension or dispersion of particles in a physiologically acceptable carrier, wherein the particles include at least one poorly soluble drug, such as a tetracycline class compound, that is chelated or otherwise complexed with a physiologically acceptable salt. The suspension/dispersion may also include at least one stabilizer and/or at least one excipient.

As used herein, the term, “drug” encompasses the free base form of a drug, as well as the corresponding salts, hydrates, solvates, prodrugs, chelates, and complexes of the drug. Thus, drugs in accordance with the present disclosure may be present, for example, in the form of a free base, a salt, a hydrate, a prodrug, a solvate (including a mixed solvate), a chelate (such as a pharmaceutically acceptable chelate with a metal salt), or a complex (such as a pharmaceutically acceptable complex, and/or a complex with a polymer).

As used herein, the term “complex” means a reversible association of compounds, molecules, atoms, etc. In contrast, the term “chelate” refers to a specific type of complex, namely a one in which a metal ion is attached to two or more bonds of the same molecule (ligands).

As used herein, the term, “poorly soluble drug,” refers to a drug that, in its neutral (i.e., uncharged) state, has a relatively low solubility in water. For example, in some embodiments of the present disclosure, the poorly soluble drug is chosen from drugs having a solubility in the neutral state at neutral pH of about 10 mg/ml or less, such as about 5 mg/ml or less, or even about 1 mg/ml or less.

As examples of poorly soluble drugs that may be used in accordance with the present disclosure, non-limiting mention is made of tetracycline class compounds, such as Doxycycline, which has a solubility of less than 10 mg/ml at neutral pH.

In some embodiments of the present disclosure, the at least one poorly soluble drug is chosen from tetracycline antibiotics. Tetracycline antibiotics include, for example, naturally-occurring and semi-synthetic, e.g. Doxycycline, Chlortetracycline, Clomocycline, Demeclocycline, Lymecycline, Meclocycline, Metacycline, Minocycline, Oxytetracycline, Penimepicycline, Rolitetracycline, and Tetracycline.

The at least one poorly soluble drug may be present in any amount suitable for a desired application. For example, the at least one poorly soluble drug may be present in an amount ranging from less than about 1% to about 90 weight %, relative to the weight of the composition. Of course, a higher or lower concentration of the at least one poorly soluble drug may be used, and the concentration may vary within the aforementioned range. For example, the poorly soluble drug may be present in an amount ranging from about 0.01% to about 90%, about 0.01% to about 10%, about 0.2 to about 5%, about <1% to about 10%, about 0.01% to about 10%, about 0.1% to about 10%, about 0.01% to about 5%, about 0.1% to about 5%, about 0.1% to about 3%, less than about 1% to about 50%, less than about 1% to about 30%, less than about 1% to about 80%, about 5% to about 90%, about 10% to about 95%, or about 0.1 to about 5% by weight, relative to the weight of the composition. In some embodiments, the at least one poorly soluble drug is present in an amount ranging from about 0.3 to about 3% by weight (e.g., about 1% by weight) relative to the weight of the composition.

The particle size of the at least one poorly soluble drug may be controlled to any desired size, so long as the particles of at least one poorly soluble drug have an average particle diameter suitable for topical application to the skin and/or a wound. For example, the particles of at least one poorly soluble drug may have an average particle size less than about 1000 μm, e.g., less than about 500 μm, less than about 300 μm, less than about 150 μm, or less than about 100 μm. Of course, particles of at least one poorly soluble drug having a larger or smaller average diameter may be used, and the average diameter may vary incrementally within the aforementioned range. In some embodiments, the particle size of the at least one poorly soluble drug ranges from about 1 to about 10 μm.

The at least one poorly soluble drug is chelated or otherwise complexed with at least one physiologically acceptable salt, e.g., a physiologically acceptable metal salt. As examples of physiologically acceptable metal salts which may be used in accordance with the present disclosure, non-limiting mention is made of calcium salts (e.g., calcium chloride) and zinc salts.

The physiologically acceptable carrier may impact the effectiveness of the at least one poorly soluble drug, and should be selected with appropriate care to ensure that a desired effectiveness of the at least one poorly soluble drug is obtained. Thus, in some embodiments of the present disclosure, the physiologically acceptable carrier is chosen from polymers, such as water-soluble polymers, polymers of neutral charge, or water-soluble polymers of neutral charge. The physiologically acceptable carrier may also be considered by the FDA to be generally regarded as safe (GRAS). As examples of physiologically acceptable carriers which may be used in accordance with the present disclosure, non-limiting mention is made of hydrogels, including cellulose containing hydrogels such as carboxy-methyl-cellulose (CMC). In some embodiments of the present disclosure, the at least one physiologically acceptable carrier also includes at least one of water, glycerol, and mixtures thereof.

The average molecular weight of the physiologically acceptable carrier may range, for example, from about 100 Daltons (Da) to about 1,000,000 Da, such as from about 500,000 Da to about 1,000,000 Da.

The viscosity of the physiologically acceptable carrier may also be chosen to suit a desired application. For example, the viscosity of the physiologically acceptable carrier may range from greater than 0 to about 10,000 centipoise (cps) or more, such as from about 100 to about 10,000 cps, from about 500 to about 5,000 cps, or even from about 1000 to about 3000 cps. In some embodiments, the physiologically acceptable carrier is a high viscosity CMC that exhibits a viscosity ranging from about 1,500 to about 3,000 cps, as measured from a 1% solution of CMC in water at 25° C. In many instances, the viscosity of the physiologically acceptable carrier is both concentration and temperature dependent. That is, the viscosity may decrease as temperature increases, and vice versa. Similarly, the viscosity may decrease as concentration decreases, and vice versa.

In some embodiments, the compositions of the present disclosure also include at least one stabilizer. Such stabilizers may serve a variety of purposes. For example, stabilizers may be added to the compositions of the present disclosure for the purpose of buffering the pH and/or the viscosity of the physiologically acceptable carrier (e.g., a hydrogel) in the presence of various metal salts. The stabilizer may be natural or synthetic, and is optionally biodegradable and/or bioerodable. Non-limiting examples of pH stabilizers that are suitable for use in accordance with the present disclosure include buffering salts and organic chemical compounds such as triethanolamine, often abbreviated as TEA, which is both a tertiary amine and a tri-alcohol. Citric acid is also suitable for use in the present disclosure as a pH stabilizer.

The compositions of the present disclosure may also include at least one excipient. The at least one excipient may be chosen, for example, from surfactants (cationic, anionic, or neutral), surface stabilizers, and other enhancers, such as preservatives. Non-limiting examples of surfactants that may be used in accordance with the present disclosure include nonionic surfactants such as a polysorbate surfactant (e.g., polysorbate 20 (Tween 20™), and polysorbate 80 (Tween 80™)). In some embodiments, the compositions of the present disclosure contain multiple pH stabilizers so as to form a pH buffering system within the composition. As an example of a preservative that may be added to the compositions of the present disclosure, non-limiting mention is made of glycerol, which may act as a preservative at certain concentrations.

The compositions of the present disclosure may also include at least one emulsifier. Non-limiting examples of suitable emulsifiers include, phospholipids, propylene glycol, polysorbate, poloxamer, and glyceryl monostearate. Of course, other known pharmaceutical emulsifiers may be used.

The compositions of the present disclosure may be in any form suitable for topical application to the skin and/or a wound. For example, the compositions may be in the form of a solution such as a hydrogel, a tincture, a cream, an ointment, a gel, a lotion, and/or an aerosol spray.

The compositions of the present disclosure may be in the form of a topical dermatologic treatment. For example, the compositions disclosed herein may be in the form of a cleansing agent, an absorbent, an anti-infective agent, an anti-inflammatory agent, an emollient (skin softener), and a keratolytic (i.e., an agent that softens, loosens, and facilitates exfoliation of the squamous cells of the epidermis).

The present disclosure also relates to methods for manufacturing compositions in accordance with the present disclosure. In some embodiments, a composition in accordance with the present disclosure is prepared by heating or autoclaving a physiologically acceptable carrier (e.g., a hydrogel), and then combining the physiologically acceptable carrier with particles of at least one poorly soluble drug (e.g., at least one tetracycline antibiotic) that is chelated or otherwise complexed with a physiologically acceptable metal salt to form a dispersion or a suspension of the physiologically acceptable carrier and the particle.

At least one stabilizer and/or at least one excipient (described previously) may be added to the physiologically acceptable carrier before or after combining the physiologically acceptable carrier with the particles. For example, a pH stabilizer such as triethanolamine may be added to the physiologically acceptable carrier to stabilize the pH of the final product and/or the dispersion/suspension, if a specific pH is desired. After the components are mixed, the final product is allowed to cool to room temperature. The viscosity of the final product may be controlled, for example, by controlling the amount of stabilizer and/or other components.

Methods of preparing the disclosed preparation may include the formation of the suspension/dispersion under high shear conditions. In addition, the suspension/dispersion may be formed using low-frequency sonication (LFS), e.g., at a frequency ranging from about 1 to about 1,000 hertz, as described in U.S. Pat. Appl. Pub. No. 2005/0175707, which is incorporated herein by reference. The use of LFS may result in improved homogeneity of the composition, relative to conventional propeller mixers or homogenizers. In addition, the size of the particles may be controlled by the intensity of the LFS as well as by controlling other conditions during the formation of the suspension/dispersion.

The composition of the present disclosure may also be present in a system for delivering an effective amount of at least one poorly soluble drug, such as a particle delivery system (“PDS”). For example, in one non-limiting embodiment, PDS includes particles of at least one poorly soluble drug, such as Doxycycline, chelated to a physiologically acceptable metal salt and dispersed and/or suspended within at least one physiologically acceptable carrier. In some embodiments, the particles of the at least one poorly soluble drug are fine particles with an average diameter of less than about 100 μm, such as about 1 to about 10 μm.

In another non-limiting embodiment, a composition or PDS in accordance with the present disclosure includes at least one hydrogel composed of at least one physiologically acceptable carrier and a solvent. As examples of suitable physiologically acceptable carriers, non-limiting mention is made of glycerol, propylene glycol, polyethylene glycol. A non-limiting example of a suitable solvent is water. Of course, other physiologically acceptable carriers and solvents may be used.

In some embodiments, the compositions and/or PDS of the present disclosure include at least one water-based hydrogel. As non-limiting examples of such hydrogels, mention is made of hydrogels prepared from polyacrylic acids, povidones, celluloses, and aloe. In some embodiments, a carboxy-methyl-cellulose hydrogel is used. Of course other hydrogels may also be used in accordance with the present disclosure.

Another aspect of the present disclosure relates to pharmaceutical formulations comprising at least one composition described herein, and/or at least one PDS comprising at least one composition described herein.

In some embodiments, the pharmaceutical formulations further comprise at least one excipient, such as a water-soluble polymer, a surfactant, and/or another enhancer such as a pharmaceutically acceptable excipient. Non-limiting examples of pharmaceutically acceptable excipients are described in Remington's Pharmaceutical Sciences by E. W. Martin, and include cellulose, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. In some embodiments, the pharmaceutical formulations also contain pH buffering reagents, and wetting or emulsifying agents.

The pharmaceutical formulations of the present disclosure can be in the any form suitable for administration to a patient, such as in the form of an aqueous dispersion or suspension. The pharmaceutical formulations may also contain various additional ingredients, such as suspending, stabilizing and/or dispersing agents.

In some embodiments, the pharmaceutical formulations described herein provide improved local concentrations of the poorly soluble drug, relative to the unformulated poorly soluble drug. For example, the local concentration may be increased by, e.g., at least 10%, 15%, 20%, 25%, 30%, 35%, 40%, 50%, 60%, 70%, 80%, 90%, 93%, 95%, 96%, 97%, 98%, 99%, 100%, 110%, 120%, 130%, 140%, 150%, or 200%, or by, e.g., at least 2, 3, 4, 5, 6, 7, 8, 9, 10, 20, 30, 40, 50, 100, or 1000 fold, as measured by, e.g., in vivo tissue distribution studies in a preclinical animal model or human clinical evaluation.

In some embodiments, the pharmaceutical formulations are in the form of a controlled-release formulation.

In some embodiments, the pharmaceutical formulations described herein are associated with improved patient compliance, relative to another pharmaceutical formulation comprising the same poorly soluble drug (which may be in another dosage form, e.g., a more invasive dosage form such as an injectable product).

Another aspect of the present disclosure relates to methods of treatment that include the topical administration of at least one composition and/or particulate delivery system in accordance with the present disclosure to the skin and/or a wound of a patient in need thereof.

As used herein, the terms “treat,” treatment,” and “treating” refer to (1) a reduction in severity or duration of a disease or condition, (2) the amelioration of one or more symptoms associated with a disease or condition without necessarily curing the disease or condition, or (3) the prevention of a disease or condition. Suitable subjects include, e.g., humans and other mammals, such as, e.g., mice, rats, dogs, and non-human primates.

In some embodiments, for example, the method includes the topical application of a composition containing an effective amount of at least one poorly soluble drug chelated to a physiologically acceptable salt (e.g., a physiologically acceptable metal salt) and dispersed and/or suspended in at least one physiologically acceptable carrier. In some instances, such a method results in beneficial (i.e., improved) wound healing, rate of wound closure, reduced inflammation, and/or reduced rate/amelioration of infection.

In some embodiments, the methods of treatment include applying a composition comprising a suspension/dispersion comprising at least one physiologically acceptable carrier (e.g., water), glycerol, wherein the suspension/dispersion comprises a physiologically acceptable carrier (e.g., a natural or synthetic polymer such as carboxy-methyl-cellulose), and at least one tetracycline antibiotic such as Doxycycline chelated to a physiologically acceptable salt, such as a calcium salt (e.g., calcium chloride). In such methods, the at least one poorly soluble drug may, for example be, present in an amount greater than about 0.1 weight % relative to the mass of the composition, such as from about 0.3% to about 1.0% by mass, or more. In some embodiments, the at least one poorly soluble drug includes Doxycycline chelated to at least one physiologically acceptable calcium salt as a fine particle suspension of particles having an average diameter less than about 100 μm (e.g., from about 1 to about 10 μm), as measured by optical microscopy.

The following examples are intended to be purely exemplary of the present invention.

### EXAMPLES

### Example 1

### Preparation of Doxycycline Monohydrate Hydrogel Particulate Delivery System

Doxycycline Monohydrate hydrogel particulate delivery systems containing 0.3, 1.0, and 3 weight % of USP grade Doxycycline Monohydrate (Spectrum Chemicals, Brunswick, N.J.), carboxy-methyl-cellulose (CMC) hydrogel, calcium chloride, glycerol, water for injection (WFI), triethanolamine, and citric acid were prepared as follows.

Briefly, a 3% CMC solution was made by mixing USP grade CMC with WFI followed by autoclaving to dissolve fully the CMC into solution, resulting in the formation of a CMC hydrogel. A Doxycycline suspension was made by adding Doxycycline Monohydrate (sieved to less than 150 μm particle size) to WFI into the CMC hydrogel. Calcium chloride and stabilizers (TEA, citric acid) were added to the CMC hydrogel, whereby calcium was available to chelate the Doxycycline. The resulting combination was mixed under high shear conditions (paddle mixer and sonication) as described in U.S. Pat. Appl. Pub. No. 2005/0175707 at elevated temperature (40 to 50 degrees Celsius). Glycerol and additional WFI were also added to the suspension. The amount of excipients added to the hydrogel was controlled to achieve a desired Doxycycline concentration.

At this dilution and temperature, the Doxycycline chelated to calcium to form a stable, small particle suspension. The 3% CMC hydrogel and Doxycycline suspension were further mixed for twenty (20) minutes, resulting in the formation of a bulk hydrogel suspension. The bulk hydrogel suspension was observed under an optical microscope at 100 to 200 times magnification. The primary particle size of the suspended particles was less then about 10 micrometers, thus permitting topical application of the composition to open wounds or other tissues without abrasion.

The final product was packaged into medical grade foil-on-foil packets in amounts suitable for the treatment of specific ailments. For example, packets were filed with a nominal 2.5 gram bulk hydrogel composition for application to small diabetic ulcers. The packaged product was then subjected to irradiation at a nominal 5 kGy. Product that passed quality control testing for content and sterility was released for use. All manufacturing steps were performed in a certified cleanroom, a laminar flow hood, or Biosafety cabinet. Standard Operating Procedures (SOPs) were followed for cleaning, gowning, material flow and testing of material.

In the event that packet filling did not occur on the same day as the synthesis of the bulk hydrogel suspension, the bulk hydrogel suspension was transferred to storage vessels (e.g., 4 L carboys), labeled, and placed into the refrigerator until packet filling was performed.

A placebo hydrogel was compounded in exactly the same manner stated above, with the exception that no Doxycyline was added to the high-shear mixer.

All hydrogels were examined for package integrity, pH, viscosity, and Total Bioburden Panel according to specified ISO, AAMI, USP, and FDA standards (method 1605000). The final product was also tested for Doxycycline content using a validated HPLC method. The result of this testing were documented on the current Production Batch Record for each lot, and final product was released for use if test results fell within acceptance limits.

### Example 2

### Diffusivity of Doxycycline from a Hydrogel stabilizer

Franz Diffusion with a permeable membrane (cellulose acetate) was used to evaluate the potential transfer of Doxycycline from the topical application of a 0.3 weight % Doxycyline Monohydrate Hydrogel and a 1.0 weight % Doxycyline Monohydrate Hydrogel manufactured in accordance with example 1 to an open wound. Static Franz diffusion cells (PermeGear) were used to obtain release data of Doxycycline from the hydrogels into a physiological buffer at 37° C. using a cellulose acetate non-rate limiting membrane (1,000 MWCO). Both the 0.3 weight % with positive control (0.3% Doxycycline in saline) and 1.0 weight % with positive control (1% Doxycycline in saline) hydrogels were analyzed for diffusion of Doxycycline across the membrane over a 24 hour period using a validated HPLC assay.

After 24 hours, less than 1% (protocol upper diffusion pass limit) of the total amount of Doxycycline available (10 mg/l gm hydrogel, in the case of the 1.0 weight % Doxycycline Monohydrate Hydrogel and 3 mg/l gm hydrogel in the case of the 0.3 weight % Doxycycline Monohydrate Hydrogel) diffused through the cellulose acetate membrane. The 0.3 weight % Doxycycline Monohydrate Hydrogel and 0.3 weight % Doxycycline Monohydrate particle suspension in saline yielded 0.12% (0.0036 mg) and 0.80% (0.02385 mg) of the 3 mg total Doxycycline available, respectively. The 1.0 weight % Doxycycline Monohydrate Hydrogel and 1.0% Doxycycline Monohydrate particle suspension in saline yielded 0.09% (0.00925 mg) and 0.37% (0.0365 mg) of the 10 mg total Doxycycline available, respectively. This data suggested that Doxycycline is retained within the local environment where the hydrogel is applied, such as an open wound.

### Example 3

### Wound Healing Study in Rats

The wound healing capability of hydrogels, containing 0.3, 1.0, and 3.0 weight % of Doxycycline Monohydrate from example 1, were tested against a placebo hydrogel (containing no Doxycycline), as well as against an untreated control. This study used full-thickness dermal punch biopsy sites to perform the evaluation of wound healing.

The results of the study showed no significant differences between the histological scoring for all concentrations and the control. Further, all hydrogel concentrations were adjudged non-irritants, relative to the controls. Additionally, it was noted that wounds treated with the Doxycycline containing hydrogels exhibited a faster healing rate (more rapid decrease in measured wound area) over the course of days 3, 7, and 10, as compared to the placebo or untreated control sites.

### Example 4

### Dermal Absorption in Rats

To measure the systemic absorption of Doxycycline from topical application of 0.3 weight %, 1.0 weight % and 3.0 weight % Doxycycline Monohydrate Hydrogels, blood samples were collected at different timepoints at Day 14 of the Wound Healing Study in example 3. Doses were approximately 20 to 200 times (0.5 to 5.25 g/kg Doxycycline for 0.3 to 3% Doxycycline hydrogels) the proposed doses for human clinical studies. Blood was collected at 30 minutes, 8 hours, 24 hours, and at sacrifice after the last test article application (Day 14). Serum was collected, frozen, and analyzed using a validated extraction/LC-MS assay.

Evaluation of Doxycycline concentration in the plasma after topical administration of the 0.3 weight %, 1.0 weight % and 3.0 weight % Doxycycline Monohydrate Hydrogels for 14 days yielded inconsistent results, ranging from 1.7 to 26.8 ng/ml for the 0.3 weight % Doxycycline Monohydrate Hydrogel, 3.2 to 73.6 ng/ml for the 1.0 weight % Doxycycline Monohydrate Hydrogel, and 2.1 to 239.5 ng/ml for the 3.0% Doxycycline Monohydrate Hydrogel. All systemic concentrations after 14 day dosing at an average of 100 times the proposed 1.0% Doxycycline Monohydrate Hydrogel dose were below all reported toxicological levels.

### Example 5

### Diabetic Foot Ulcer Study in Humans

In a small IRB-approved human clinical study, six diabetic patients were given topical doxycycline hydrogel compared to placebo until the ulcer healed. Wounds treated with Doxycycline healed, in contrast, only one of the three patients treated with placebo healed during the initial 20 week treatment period. Statistical analysis of the healing outcome of the patients at 34 weeks indicated that topical Doxycycline hydrogel treatment significantly increased healing of the ulcers compared to treatment with placebo hydrogel.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A composition for topical application to skin or a wound, comprising a suspension or dispersion of particles of at least one tetracycline class compound complexed with a physiologically acceptable metal salt, wherein the composition further comprises at least one physiologically acceptable carrier, at least one excipient, and at least one stabilizer, and said particles have an average diameter less than or equal to about 100 μm.

2. The composition of claim 1, wherein said physiologically acceptable metal salt comprises a physiologically acceptable calcium salt.

3. The composition according to claim 2, wherein said at least one tetracycline class compound is chelated to said physiologically acceptable calcium salt.

4. The composition of claim 1, wherein said at least one tetracycline class compound is chosen from tetracycline antibiotics.

5. The composition of claim 4, wherein said at least one tetracycline class compound is chosen from Doxycycline, Chlortetracycline, Clomocycline, Demeclocycline, Lymecycline, Meclocycline, Metacycline, Minocycline, Oxytetracycline, Penimepicycline, Rolitetracycline, and Tetracycline, and salts, hydrates, chelates, and complexes thereof.

6. The composition of claim 4, wherein said at least one tetracycline class compound is chosen from Doxycyclene and the salts, hydrates, chelates, and complexes thereof.

7. The composition of claim 6, wherein said Doxycycline is present in said composition in an amount ranging from greater than about 0.3 weight % to about 3 weight %.

8. The composition of claim 1, wherein said at least one physiologically acceptable carrier is chosen from water soluble polymers, polymers of neutral charge, and water-soluble polymers of neutral charge.

9. The composition of claim 8, wherein said at least one physiologically acceptable carrier is a hydrogel.

10. The composition of claim 9, wherein said hydrogel comprises cellulose.

11. The composition of claim 1, wherein said particles have an average particle diameter ranging from about 1 to about 10 μm.

12. The composition of claim 1, wherein said at least one excipient is chosen from surfactants, surface stabilizers, and preservatives.

13. The composition of claim 12, wherein said surfactants comprise at least one nonionic surfactant, and said preservatives comprise glycerol.

14. The composition of claim 1, wherein said at least one stabilizer comprises at least one pH stabilizer comprising at least one of buffering salts, triethanolamine, and citric acid.

15. A composition for topical application to skin or a wound, comprising a suspension or dispersion of particles of at least one tetracycline class compound chelated to a physiologically acceptable calcium salt, wherein the composition further comprises a carboxy-methyl-cellulose hydrogel, glycerol, water, and at least one pH stabilizer, and said particles have an average diameter less than or equal to about 100 μm.

16. A method of making a composition for topical application to skin and/or a wound, comprising:
combining at least one tetracycline class compound with at least one physiologically acceptable metal salt to form metal-chelated particles;
forming said metal-chelated particles and at least one physiologically acceptable carrier into a suspension or dispersion of metal-chelated particles, said particles having an average diameter less than or equal to about 100 μm; and
optionally combining at least one excipient and/or at least one stabilizer with said suspension or dispersion of metal-chelated particles.

17. The method of claim 16, wherein said physiologically acceptable metal salt comprises a physiologically acceptable calcium salt.

18. The method of claim 16, wherein said at least one tetracycline class compound is chosen from tetracycline antibiotics.

19. The method of claim 18, wherein said at least one tetracycline class compound is chosen from Doxycycline, Chlortetracycline, Clomocycline, Demeclocycline, Lymecycline, Meclocycline, Metacycline, Minocycline, Oxytetracycline, Penimepicycline, Rolitetracycline, and Tetracycline, and salts, hydrates, chelates, and complexes thereof.

20. The method of claim 19, wherein said at least one tetracycline class compound is chosen from Doxycycline and the salts, hydrates, chelates, and complexes thereof.

21. The method of claim 20, wherein said Doxycycline is present in said composition in an amount ranging from greater than about 0.01 weight % to about 3 weight %.

22. The method of claim 16, wherein said at least one physiologically acceptable carrier is chosen from water soluble polymers, polymers of neutral charge, and water-soluble polymers of neutral charge.

23. The method of claim 22, wherein said at least one physiologically acceptable carrier is a hydrogel.

24. The method of claim 23, wherein said hydrogel comprises cellulose.

25. The method of claim 16, comprising combining said suspension or dispersion of metal-chelated particles with at least one excipient comprising at least one of a surfactant, surface stabilizer, and preservative.

26. The method of claim 25, wherein said surfactants comprise at least one nonionic surfactant, and said preservatives comprise at least one of glycerol, citric acid, and a mixture thereof.

27. The method of claim 16, comprising combining said suspension or dispersion of metal-chelated particles with at least one stabilizer, wherein said at least one stabilizer comprises at least one pH stabilizer chosen from buffering salts, triethanolamine, and citric acid.

28. A method of treating skin or a wound, comprising, providing a pharmaceutical formulation comprising an effective amount of a composition according to claim 1, and topically administering said pharmaceutical formulation to skin or a wound on a patient in need thereof.
